(19) United States
(12) Patent Application Publication
(10) Pub. No.: US 2025/0039542 A1
(43) Pub. Date: Jan. 30, 2025

(54) ANIMAL IN-VIVO IMAGING DEVICE AND OPERATING METHOD THEREOF

(71) Applicant: Vieworks Co., Ltd., Anyang-si (KR)

(72) Inventors: Yeong Hyeon PYEON, Anyang-si (KR); Dong-Uk LEE, Gwacheon-si (KR); Mingyu JIN, Seoul (KR)

(21) Appl. No.: 18/915,923

(22) Filed: Oct. 15, 2024

Related U.S. Application Data
(63) Continuation of application No. PCT/KR2023/002518, filed on Feb. 22, 2023.

(30) Foreign Application Priority Data
Apr. 13, 2022 (KR) 10-2022-0045784

(51) Int. Cl. H04N 23/67; A61B 5/00; G06T 7/00; G06T 7/50; G06T 7/70; H04N 7/18

(52) U.S. Cl. CPC H04N 23/67; A61B 5/0064; A61B 5/0071; A61B 5/7267; A61B 5/7425; G06T 7/0012; G06T 7/50; G06T 7/70; H04N 7/181; A61B 2503/40; G06T 2200/04; G06T 2207/10064; G06T 2207/20081; G06T 2207/20084; G06T 2207/30004

(57) ABSTRACT

An animal in-vivo imaging device comprises: a camera for capturing an image of an animal and including a focus lens for adjusting a focus; a three-dimensional scanner for measuring three-dimensional shape information of the animal; an estimation unit configured to output depth information of a target organ by inputting the type of animal, the target organ, a preview image captured by the camera, and the three-dimensional shape information measured by the three-dimensional scanner into a trained neural network model; and a focus adjustment unit that adjusts a focus by controlling a focus driving motor that drives the focus lens according to the depth information.

### CROSS-REFERENCE TO RELATED APPLICATION

The present application is a continuation of PCT/KR2023/002518, filed on Feb. 22, 2023, which claims priority to Korean Patent Application No. 10-2022-0045784, filed on Apr. 13, 2022, the entire contents of which are incorporated herein for all purposes by this reference.

### TECHNICAL FIELD

The present invention relates to an animal in-vivo imaging device and an operating method thereof.

### BACKGROUND ART

In general, an animal in-vivo imaging device refers to an imaging device that analyzes bioluminescence or fluorescence signals of small animals such as a laboratory mouse to study the anatomical structure and function, or to measure and evaluate the pharmacological response of cells. The animal in-vivo imaging device can noninvasively obtain images of internal animal organs by capturing an animal with a fluorescent substance injected into the organ to be observed using a camera.

To obtain optimal images using an animal in-vivo imaging device, an area to be captured must be located within the depth of field range. In general, a focusing method of a camera sets a focus value specified for each field of view (FOV) or performs a focus using an autofocus function. However, since the types and sizes of animals to be captured (such as mice and rats) are diversified, the animal's posture and capturing area are different, and the depth of field changes depending on a magnification ratio, it is difficult to focus on various capturing subjects using the set focus value or the autofocus function.

FIG. 1 illustrates an example of an out-of-focus case during in-vivo imaging of an animal. Referring to part (a) of FIG. 1, a small animal enters a depth of field range according to a default set focus value, but in the case of a large animal, a partial area deviates from the depth of field range. Referring to part (b) of FIG. 1, in the case of focusing through the autofocus function, a distance to the surface of a subject varies, so for example, when focusing on the widest range, an intended location, such as an organ, deviates from the depth of field.

### DISCLOSURE

### Technical Problem

A technical object to be achieved by the present invention is to provide an animal in-vivo imaging device and an operating method thereof which are capable of obtaining an optimal image by automatically adjusting a focus of a camera according to a subject to be captured and an organ to be captured.

### Technical Solution

In order to achieve the technical object, an animal in-vivo imaging device according to the present invention includes: a camera for capturing an image of an animal and including a focus lens for focus adjustment; a three-dimensional scanner for measuring three-dimensional shape information of the animal; an estimation unit inputting the type of animal, a target organ, a preview image captured by the camera, and three-dimensional shape information measured by the three-dimensional scanner into a trained neural network model to output depth information of the target organ; and a focus adjustment unit adjusting a focus by controlling a focus driving motor which drives the focus lens according to the depth information, and the neural network model is trained by using a training dataset which uses, as inputs, the type of animal, the target organ, the captured image of the animal, and the three-dimensional shape information, and uses, as an output, depth information of the target organ.

The estimation unit may further output horizontal position information of the target organ, and the training dataset may further have the horizontal position information of the target organ as an output.

The animal in-vivo imaging device may further include an image display unit displaying and outputting the position information in the captured image in which the focus is adjusted by the focus adjustment unit.

The animal in-vivo imaging device may further include a retraining unit retraining, when a part which appears fluorescence in the captured image and the output position information do not match, the neural network model by using training data which has position information of the part which appears the fluorescence as an output.

The estimation unit may further input the posture of the animal into the neural network model, and the training dataset may further use the posture of the animal as an input.

In order to achieve the technical object, an operating method of an animal in-vivo imaging device including a camera for capturing an image of an animal and including a focus lens for focus adjustment, and a three-dimensional scanner for measuring three-dimensional shape information of the animal according to the present invention includes: inputting the type of animal, a target organ, a preview image captured by the camera, and three-dimensional shape information measured by the three-dimensional scanner into a trained neural network model to output depth information of the target organ; and adjusting a focus by controlling a focus driving motor which drives the focus lens according to the depth information, and the neural network model is trained by using a training dataset which use, as inputs, the type of animal, the target organ, the captured image of the animal, and the three-dimensional shape information, and uses, as an output, depth information of the target organ.

In the outputting, horizontal position information of the target organ may be further output, and the training dataset may further have the horizontal position information of the target organ as an output.

The operating method of an animal in-vivo imaging device may further include displaying and outputting the position information in the captured image in which the focus is adjusted by the adjusting of the focus.

The operating method of an animal in-vivo imaging device may further include retraining, when a part which appears fluorescence in the captured image and the output position information do not match, the neural network model by using training data which has position information of the part which appears fluorescence as an output.

In the outputting, the posture of the animal may be further input into the neural network model, and the training dataset may further use the posture of the animal as an input.

### Advantageous Effects

According to the present invention, a focus of a camera is automatically adjusted according to an animal to be captured and an organ to be captured by an animal in-vivo imaging device to obtain an optimal image.

### DESCRIPTION OF DRAWINGS

FIG. 1 illustrates an example of an out-of-focus case during in-vivo imaging of an animal.

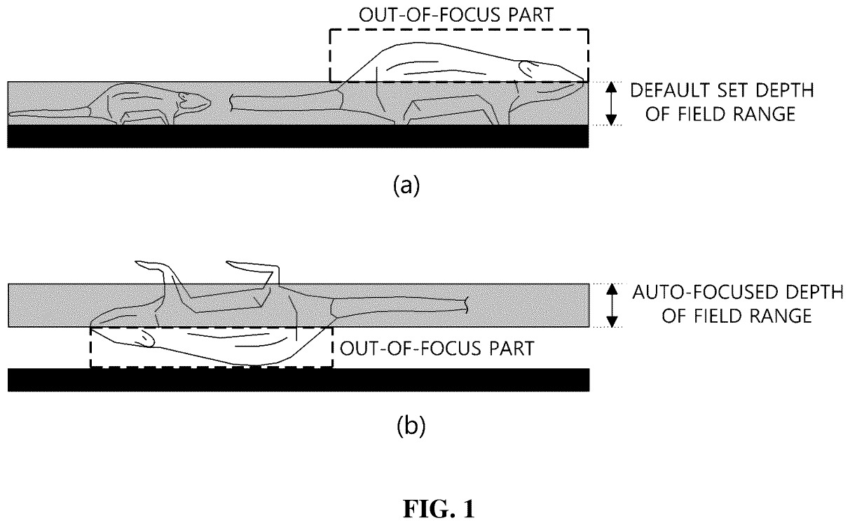

FIG. 2 illustrates a configuration of an animal in-vivo imaging device according to an embodiment of the present invention.

FIG. 3 illustrates an example of a neural network model.

FIG. 4 illustrates a flowchart of a method for training a neural network model.

FIG. 5 shows an example of a process in which a training dataset is prepared.

FIG. 6 illustrates a flowchart of an operating method of an animal in-vivo imaging device according to an embodiment of the present invention.

FIG. 7 shows an example of an operation of an animal in-vivo imaging device according to an embodiment of the present invention.

### MODE FOR INVENTION

Hereinafter, preferred embodiments of the present invention will be described in detail with reference to the drawings. In the following descriptions and the accompanying drawings, substantially the same components are represented by the same reference numerals, and the duplicate description will be omitted. Further, in describing the present invention, a detailed explanation of a known related function or configuration may be omitted to avoid unnecessarily obscuring the subject matter of the present invention.

A vertical depth and a horizontal position of an animal's organs within the body will be closely related to the animal's type, size, and overall shape. Accordingly, the inventor of the present application conceived the present invention based on the idea that if there is sufficient data on the depth and position of organs according to the type, size, and overall shape of an animal, the depth and location of the organs can be estimated through an artificial intelligence model, and the focus of the camera can be adjusted accordingly.

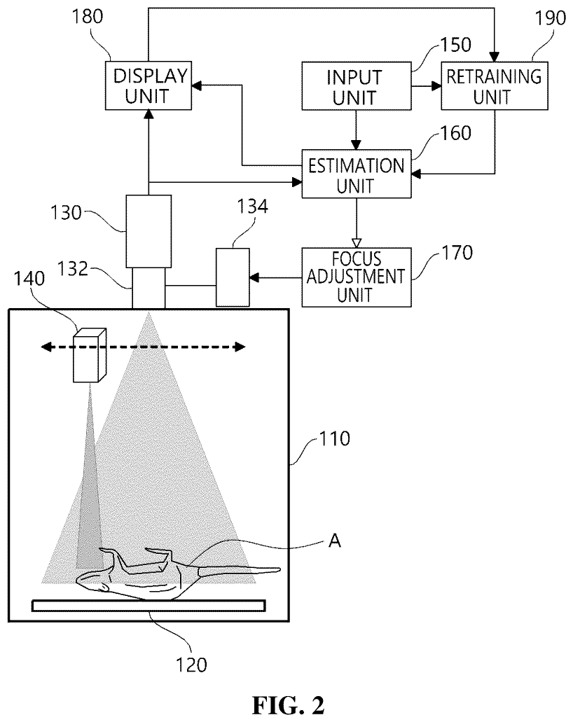

FIG. 2 illustrates a configuration of an animal in-vivo imaging device according to an embodiment of the present invention.

The animal in-vivo imaging device according to an embodiment of the present invention may include a darkroom box 110, a stage 120, a camera 130, a focus driving motor 134, a three-dimensional scanner 140, an input unit 150, an estimation unit 160, a focus adjustment unit 170, a display unit 180, and a retraining unit 190.

The darkroom box 110 may be formed into various shapes so as to accommodate an animal A while blocking external light. The stage 120 may be installed inside the darkroom box 110 so that the animal A may be placed thereon.

The animal A may include a mouse, a rat, a rabbit, a dog, a cat, a pig, etc., which are mainly used for research. The animal A may be placed on the stage 120 while adopting a material applied thereto designed to cause fluorescence to be emitted in an organ to be observed.

The camera 130 is installed to capture the animal A placed on the stage 120. The camera 130 may be equipped with a focus lens 132 for focus adjustment. The focus of the camera 130 may be adjusted by driving the focus lens 132 by the focus driving motor 134.

The three-dimensional scanner 140 is installed to measure three-dimensional shape information of the animal A placed on the stage 120. The three-dimensional scanner 140 may be a laser-type or optical (white light)-type three-dimensional scanner. The three-dimensional scanner 140 may measure the three-dimensional shape information, for example, using a line scanning scheme, or measure the entire area at once using a fixation scheme.

The input unit 150 receives, from a user, the type of animal A, a posture of the animal A, and a target organ to be observed as inputs. The posture of the animal A may include, for example, ‘face down’, where a face is facing downward, and ‘face up’, where the face is facing upward. The target organ may include, for example, the brain, heart, lungs, stomach, liver, kidney, colon, small intestine, rectum, bladder, etc.

The estimation unit 160 inputs the type of animal A, the posture of the animal A, the target organ, a preview image of the animal A captured by the camera 130, and the three-dimensional shape information of the animal A measured by the three-dimensional scanner 140 into a trained neural network model, thereby outputting vertical depth information and horizontal position information of the target organ estimated through the neural network model. Here, the depth information may represent a vertical distance between the surface of the animal A and the target organ, and the position information may represent the position of the center of the target organ or the area of the target organ in the preview image of the animal A (or an image obtained by cropping the area of the animal A from the preview image).

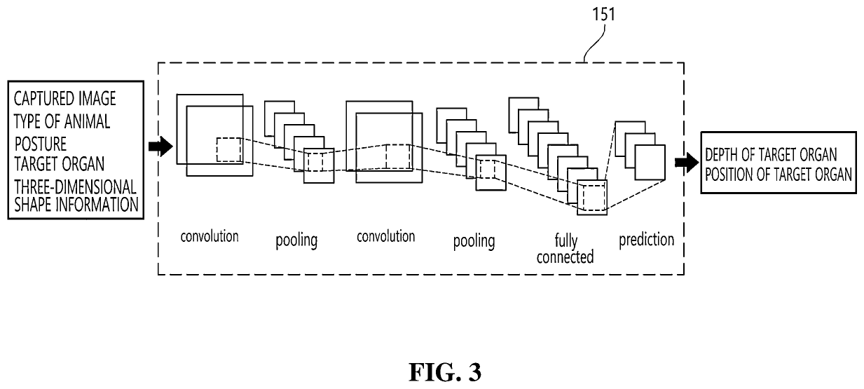

FIG. 3 illustrates an example of a neural network model 151 used in the estimation unit 160. Referring to FIG. 3, the neural network model 151 may use, as inputs, a captured image of the animal, the type of animal, the posture of the animal, the target organ, and three-dimensional shape information, and use, as outputs, depth information and position information of the target organ. The neural network model 151 as a convolutional neural network may be constituted by, for example, multiple convolutional layers, a pooling layer, a fully connected layer, a prediction layer, etc.

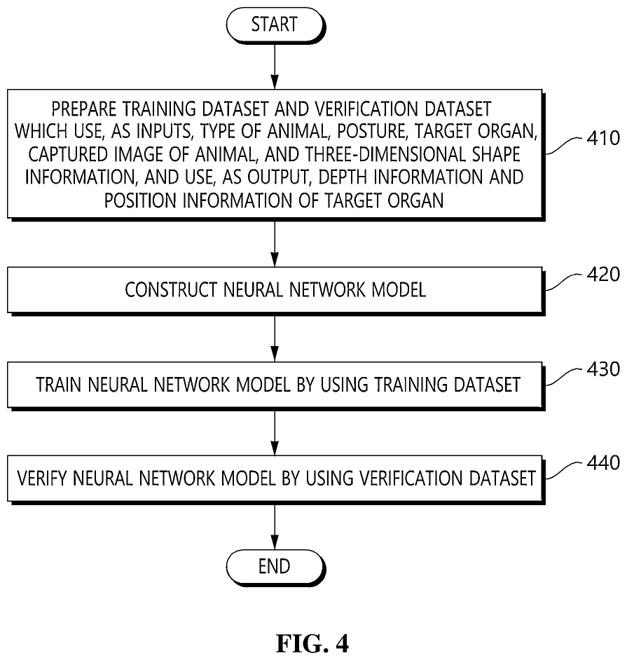

FIG. 4 illustrates a flowchart of a method for training the neural network model 151.

In step 410, a training dataset and a verification dataset are prepared which have the type of animal, the posture of the animal, the target organ, the captured image of the animal, and the three-dimensional shape information as the input, and have the depth information and the position information of the target organ as the input. Here, the depth information and the position information of the target organ may be collected by referring to a past capturing history or by using anatomical information of the animal.

In step 420, the neural network model 151 is constructed, which has, as the input, the captured image of the animal, the type of animal, the posture of the animal, the target organ, and the three-dimensional shape information, and as, the output, the depth information and the position information of the target organ.

In step 430, the neural network model 151 is trained using the training dataset. Through training, a weight, a bias, etc. of the neural network model 151 may be adjusted. A gradient descent algorithm, a back propagation algorithm, etc., may be used as a neural network learning technique.

In step 440, the neural network model 151 is verified using the verification dataset. When verification fails, the neural network model 151 may be modified or transformed, or may be retrained by reinforcing the training dataset.

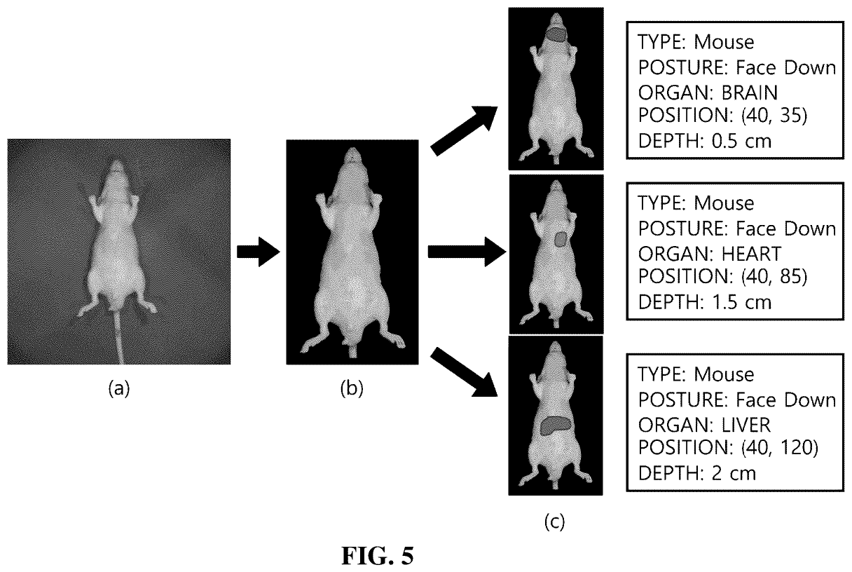

FIG. 5 shows an example of a process in which a training dataset is prepared. Referring to FIG. 5, in an animal capturing image (part (a) of FIG. 5), an area of the animal is cropped (part (b) of FIG. 5), a portion of each target organ such as the brain, heart, and liver is marked (part (c) of FIG. 5), and the type of animal, posture, location and depth of the target organ may be set. Further, the three-dimensional shape information may be measured for the animal using the three-dimensional scanner.

Referring back to FIG. 2, the focus adjustment unit 170 controls the focus driving motor 134 according to the depth information output from the estimation unit 160 to adjust the focus of the camera 130 to the target organ. For example, the focus adjustment unit 170 may focus the camera 130 on the surface of the animal A, and then move the focus by the depth information of the target organ to focus on the target organ by using the autofocus function.

The display unit 180 may display the position information output from the estimation unit 160 in a captured image by adjusting the focus by the focus adjustment unit 170. In the captured image of the animal A, a target organ portion appears fluorescence, but a fluorescence area may appear over a wider area than the target organ portion, displaying the position information of the target organ may be helpful for organ observation.

Meanwhile, when a part that appears fluorescence in the captured image of the animal A and the position information output from the estimation unit 160 do not match, it can be seen that there is an error in position estimation of the target organ of the neural network model 151. Therefore, in this case, the retraining unit 190 may retrain the neural network model 151 by using training data that inputs the type, posture, and target organ of the animal A input through the input unit 150, and the preview image of the animal A captured by the camera 130, and outputs the position information of the part that appears fluorescence in the captured image.

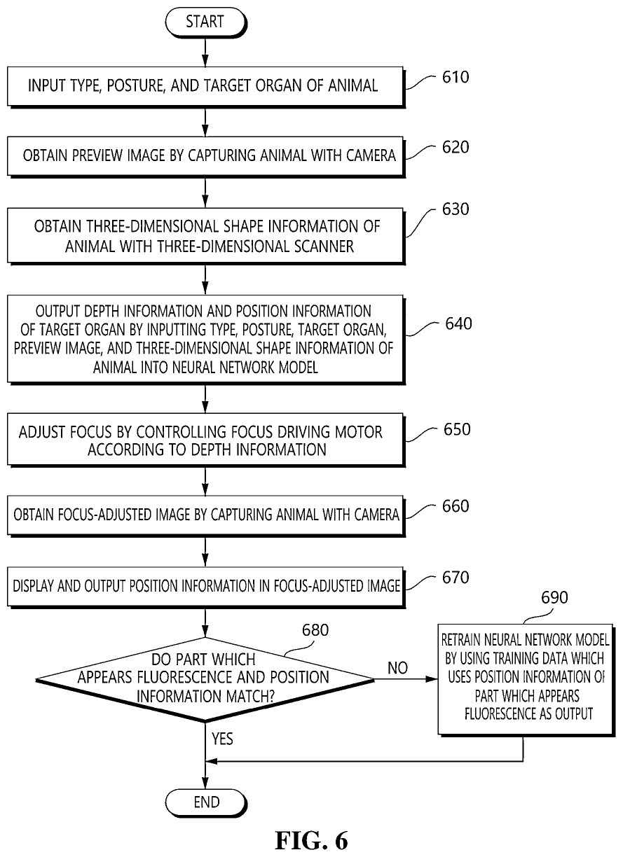

FIG. 6 illustrates a flowchart of an operating method of an animal in-vivo imaging device according to an embodiment of the present invention.

In step 610, the type of animal A, a posture of the animal A, and a target organ to be observed as an input are received from a user.

In step 620, a preview image is obtained by capturing the animal A with a camera 130. The preview image may be provided as an image obtained by cropping an area of the animal A from the captured image of the animal A.

In step 630, three-dimensional shape information of the animal A is obtained with a three-dimensional scanner 140.

In step 640, the type of animal A, the posture of the animal A, the target organ, the preview image, and the three-dimensional shape information are input into the neural network model 151 to output depth information and position information of the target organ.

In step 650, a focus driving motor 134 is controlled according to the depth information to adjust a focus of the camera 130 to be focus the target organ.

In step 660, a focus-adjusted fluorescent image is obtained by fluorescence-capturing the animal A with the camera 130. A fluorescent image in which a fluorescent area appears normally may be obtained through focus adjustment.

In step 670, position information is displayed in the focus-adjusted fluorescent image, which is output. In this case, the fluorescent image and the preview image may be overlaid and output.

In step 680, when the part that appears fluorescence in the captured image of the animal A does not match the position information, in step 690, the retraining unit 190 may retrain the neural network model 151 using training data that outputs the position information of the part that appears fluorescence.

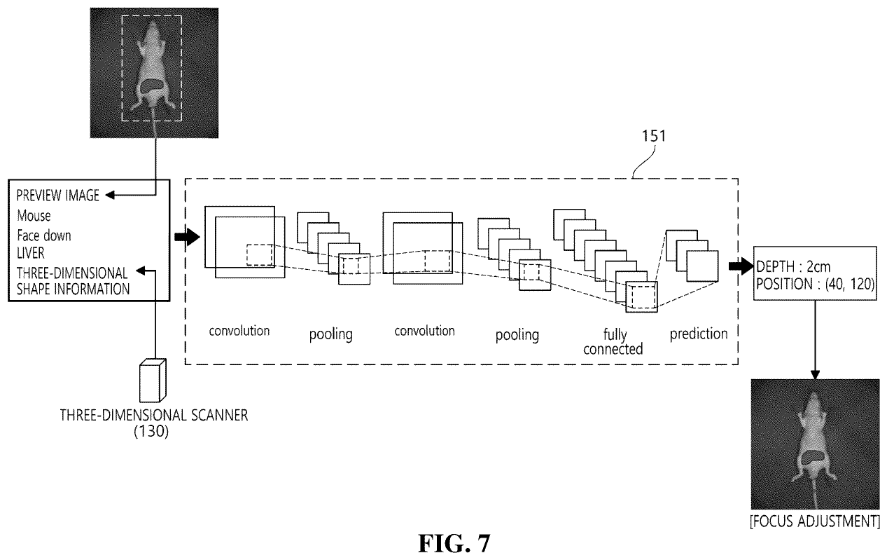

FIG. 7 shows an example of an operation of an animal in-vivo imaging device according to an embodiment of the present invention.

When a preview image captured by the camera 130, the type of animal (mouse) input from the user, a posture (face down), a target organ (liver), and three-dimensional shape information measured by the three-dimensional scanner 140 are input into the neural network model 151, the neural network model 151 outputs depth information (2 cm) and position information (40, 120) for the liver of the animal (mouse). Then, the focus driving motor 134 is controlled according to the depth information (2 cm) to obtain an image in which a liver portion is focused.

A device according to embodiments of the present invention may include a processor, a memory for storing and executing program data, a permanent storage such as a disk drive, a communication port for communicating with an external device, a user interface device such as a touch panel, a key, a button, and the like. Methods implemented as software modules or algorithms may be stored on a computer-readable recording medium as computer-readable codes or program instructions executable on the processor. Here, computer-readable recording media include magnetic storage media (e.g., read-only memory (ROM), random-access memory (RAM), floppy disks, hard disks, etc.) and optical reading media (e.g., CD-ROM, DVD: Digital Versatile Disc). The computer readable recording media may be stored and executed as codes which may be distributed in the computer system connected through a network and read by a computer in a distribution method. The media are readable by the computer, and may be stored in the memory and executed by the processor.

Embodiments of the present invention may be represented by functional block configurations and various processing steps. These functional blocks may be implemented as various numbers of hardware and/or software components that perform specific functions. For example, embodiments may employ integrated circuit components, such as memory, processing, logic, look-up tables, etc., that may perform various functions under the control of one or more microprocessors or other control devices. The components of the present invention may be implemented as software programming or software elements, and similarly, embodiments may include various algorithms implemented as combinations of data structures, processes, routines or other programming components to be implemented in a programming or scripting language such as C, C++, Java, assembler, etc. Functional aspects may be implemented as algorithms executed on one or more processors. Additionally, the embodiments may employ conventional techniques for electronic environmental configuration, signal processing, and/or data processing. Terms such as “mechanism”, “element”, “means”, and “component” may be used broadly and are not limited to mechanical and physical components. The above term may include the meaning of a series of software processes (routines) in connection with a processor, etc.

Specific executions described in the embodiment are exemplary embodiments and the scope of the embodiment is not limited even by any method. For brevity of the specification, descriptions of conventional electronic configurations, control systems, software, and other functional aspects of the systems may be omitted. Further, connection or connection members of lines among components illustrated in the drawing exemplarily represent functions connections and/or physical or circuitry connections and may be represented as various functional connections, physical connections, or circuitry connections which are replaceable or added in an actual device. Further, unless otherwise specified, such as “essential”, “important”, etc., the connections may not be components particularly required for application of the present invention.

The present invention has been described above with reference to preferred embodiments thereof. It is understood to those skilled in the art that the present invention may be implemented as a modified form without departing from an essential characteristic of the present invention. Therefore, the disclosed embodiments should be considered in an illustrative viewpoint rather than a restrictive viewpoint. The scope of the present invention is defined by the appended claims rather than by the foregoing description, and all differences within the scope of equivalents thereof should be construed as being included in the present invention.

What is claimed is:

1. An animal in-vivo imaging device comprising:
a camera for capturing an image of an animal and including a focus lens for focus adjustment;
a three-dimensional scanner for measuring three-dimensional shape information of the animal;
an estimation unit configured to output depth information of the target organ by inputting the type of animal, a target organ, a preview image captured by the camera, and three-dimensional shape information measured by the three-dimensional scanner into a trained neural network model; and
a focus adjustment unit configured to adjust a focus by controlling a focus driving motor which drives the focus lens according to the depth information,
wherein the neural network model is trained by using a training dataset which uses, as inputs, the type of animal, the target organ, the captured image of the animal, and the three-dimensional shape information, and uses, as an output, depth information of the target organ.

2. The animal in-vivo imaging device of claim 1, wherein the estimation unit further outputs horizontal position information of the target organ, and
the training dataset further has the horizontal position information of the target organ as an output.

3. The animal in-vivo imaging device of claim 2, further comprising:
an image display unit configured to display and output the position information in the captured image in which the focus is adjusted by the focus adjustment unit.

4. The animal in-vivo imaging device of claim 2, further comprising:
a retraining unit configured to retrain, when a part which appears fluorescence in the captured image and the output position information do not match, the neural network model by using training data which has position information of the part which appears fluorescence as an output.

5. The animal in-vivo imaging device of claim 1, wherein the estimation unit further inputs the posture of the animal into the neural network model, and
the training dataset further uses the posture of the animal as an input.

6. An operating method of an animal in-vivo imaging device including a camera for capturing an image of an animal and including a focus lens for focus adjustment, and a three-dimensional scanner for measuring three-dimensional shape information of the animal, comprising:
inputting the type of animal, a target organ, a preview image captured by the camera, and three-dimensional shape information measured by the three-dimensional scanner into a trained neural network model to output depth information of the target organ; and
adjusting a focus by controlling a focus driving motor which drives the focus lens according to the depth information,
wherein the neural network model is trained by using a training dataset which uses, as inputs, the type of animal, the target organ, the captured image of the animal, and the three-dimensional shape information, and uses, as an output, depth information of the target organ.

7. The operating method of an animal in-vivo imaging device of claim 6, wherein in the outputting,
horizontal position information of the target organ is further output, and
the training dataset further has the horizontal position information of the target organ as an output.

8. The operating method of an animal in-vivo imaging device of claim 7, further comprising:
displaying and outputting the position information in the captured image in which the focus is adjusted by the adjusting of the focus.

9. The operating method of an animal in-vivo imaging device of claim 7, further comprising:
retraining, when a part which appears fluorescence in the captured image and the output position information do not match, the neural network model by using training data which has position information of the part which appears fluorescence as an output.

10. The operating method of an animal in-vivo imaging device of claim 6, wherein in the outputting,
the posture of the animal is further input into the neural network model, and
the training dataset further uses the posture of the animal as an input.
